(19) United States
(12) Patent Application Publication
(10) Pub. No.: US 2023/0225893 A1
(43) Pub. Date: Jul. 20, 2023

(54) HEALTH BRACE

(71) Applicant: 3M INNOVATIVE PROPERTIES COMPANY, St. Paul, MN (US)

(72) Inventors: Nicholas K. Lee, Minneapolis, MN (US); Diane L. Emslander, Grant, MN (US); Dale R. Wiehe, Lake Elmo, MN (US)

(21) Appl. No.: 17/917,341

(22) PCT Filed: Apr. 20, 2021
(86) PCT No.: PCT/IB2021/053264

§ 371 Date: Oct. 6, 2022

Related U.S. Application Data
(60) Provisional application No. 63/013,419, filed on Apr. 21, 2020.

(51) Int. Cl. A61F 5/01

(52) U.S. Cl. CPC A61F 5/0104

(57) ABSTRACT

The present invention is brace including a main body and a strap attached to the main body for adjusting the main body around a body part. The main body and the strap are a unitary body. The brace includes a first zone having a first specific elongation and a second zone having a second specific elongation.

### FIELD OF THE INVENTION

The present invention relates to a health brace. In particular, the present invention is a brace designed to provide support, comfort, and fit.

### BACKGROUND OF THE INVENTION

There are currently two general types of braces and supports to help manage or alleviate chronic or acute pain. Throughout this specification, the term “brace” is meant to be used generally and also includes supports. The first type of brace is formed from different tri-laminates and/or materials that are pieced together by sewing to provide differentiated compression or support. The amount of support and compression can be modified based on the materials forming the tri-laminate and how the various materials and tri-laminates are positioned relative to each other in the finished, sewn brace. Many of these braces utilize tri-laminates that are made up of a polyurethane foam or neoprene core layer with fabrics and/or unbroken loops as the exterior layers. Oftentimes, these braces include straps to provide adjustability, allowing for custom support, comfort, and fit to the user.

The second type of brace is formed from a single piece of material, usually a flat knit or circular knit. In some embodiments, the single piece brace can provide graduated compression or different zones of compression. However, this type of brace is usually a form similar to a sleeve or a sock that cannot be adjusted to customize support, comfort, or fit.

### SUMMARY OF THE INVENTION

In one embodiment, the present invention is a brace including a main body and a strap attached to the main body for adjusting the main body around a body part. The main body and the strap are a unitary body. The brace includes a first zone having a first specific tensile response and a second zone having a second specific tensile response.

In another embodiment, the present invention is a brace including a body having a core layer and a strap attached to the body. The body includes a first zone having a first specific tensile response and a second zone having a second specific tensile response. The core layer has a thickness of no greater than about 0.065 inches.

### BRIEF DESCRIPTION OF THE FIGURES

This disclosure may be more completely understood in consideration of the following detailed description of various embodiments of the disclosure in connection with the accompanying drawings, in which:

FIGS. 1-7 show various embodiments and views of a brace of the present invention.

FIGS. 8(a), 8(b), and 8(c) are schematics showing the laser perforation paths for various embodiments of the present invention.

FIGS. 9(a), 9(b), 9(c), and 9(d) are schematics showing the laser slitting patterns for various embodiments of the present invention.

FIG. 10 is a drawing of examples of the present invention, showing the lamination area.

FIG. 11 is a schematic of a welding template used to prepare various embodiments of the present invention.

FIG. 12 shows examples of the present invention after and during tensile testing.

While the above-identified figures set forth several embodiments of the disclosure, other embodiments are also contemplated, as noted in the description. In all cases, this disclosure presents the invention by way of representation and not limitation. It should be understood that numerous other modifications and embodiments can be devised by those skilled in the art, which fall within the scope and spirit of the principles of the invention.

### DETAILED DESCRIPTION

The present invention is an adjustable brace that provides support, comfort, and fit for users of various sizes. The brace provides at least two zones of specific tensile response with differentiated levels of compression, elasticity, and support provided in each zone. In one embodiment, the brace is a unitary piece. This has the benefit of increasing comfort as there are no seams, as well as simplifying the production process. In one embodiment, the brace has a maximum thickness, allowing for a lightweight support that can be easily worn under clothing and allowing for a wide range of motion in an athletic setting. The construction of the brace thus provides customized compression with targeted support zones and expansion zones for dynamic fit and comfort, while maintaining adjustability, breathability, and a low profile.

Support within the context of a brace is a combination of applied compression and proprioceptive stabilization across the joint range of movement. Depending on the joint, this may require materials that are inelastic. Fit requires materials to stretch in specific areas to either conform to the complex curvature of the human anatomy or to have an adjustable length to accommodate the size range of potential users. Comfort generally means that the brace feels soft to the touch, provides even contact pressure with a low shear friction across an entire joint range of movement, and allows adequate dissipation of heat and moisture.

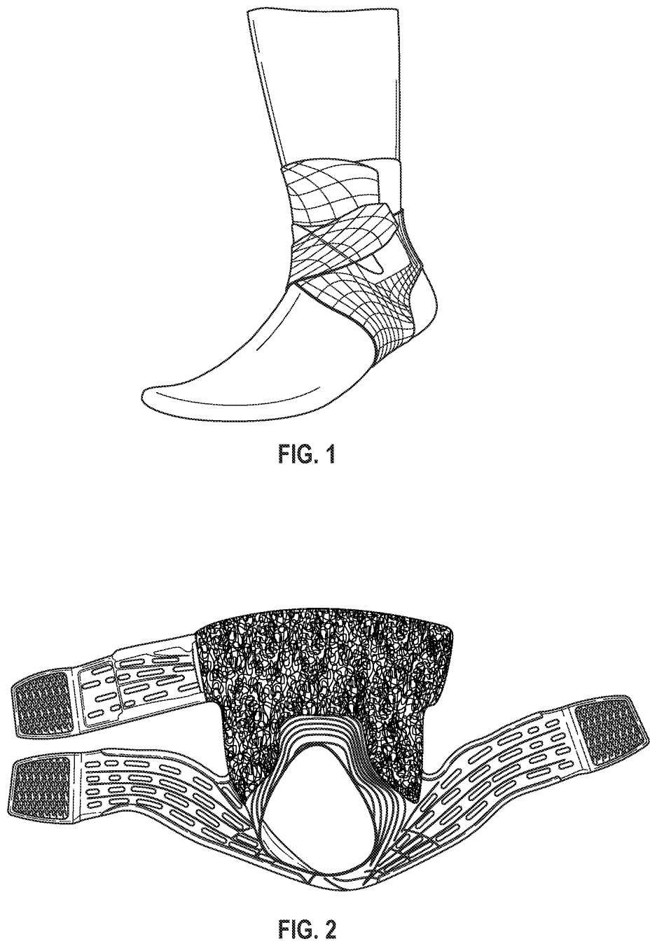

Various embodiments and views of the brace of the present invention are shown in FIGS. 1, 2, 3, 4, 5, 6, and 7. While FIGS. 1-7 specifically show an ankle brace for use on a user's ankle, the construction of the brace of the present invention can be used for any brace designed for any specific number of body parts without departing from the intended scope of the present invention. For example, the brace construction can be used for body parts including, but not limited to: ankles, wrists, knees, shoulders, elbows, and backs. In addition, while the present invention is described as a brace or a support, the construction of the present invention can be used not only as a health brace but also as apparel or footwear.

The brace of the present invention includes a main body and at least one strap extending from the main body. The strap includes a first end and a second end and functions to maintain the brace to a body part. The first end of the strap extends from the main body while the second end can either be free or attached to the brace. The strap is repositionable with the second end attachable to either the main body or to the strap itself. In practice, the strap provides adjustability to the brace, allowing the user to customize the support, comfort, and fit of the brace on the body part.

In one embodiment, the brace made up of the main body and the strap is a unitary piece and is formed of at least a core layer. By unitary, it is meant that the brace is not formed of cut and seamed pieces, but rather, is formed from a single piece of core layer. In one embodiment, the brace uses non-woven input material for fabrication. In one embodiment, the core layer is a foam formed of a multi-layered polymeric material. In one embodiment, the core layer is made using blown film, or a tube that is collapsed to form a single layer. An example of the core layer includes, but is not limited to, the following:

| Material | Function |
|---|---|
| Elastollan 1170A TPU | High Elastic Recovery + |
| | Enables RF Welding |
| Bynel 3860 | Tie Layer to Bond TPU to the Core |
| Vistamaxx 6102 (PP) + | Low Cost Foam − |
| 2%-10% Expandable | Good Elastic Properties |
| Microspheres (Akzo 950 MB 80) | |
| Versaflex MD6748 | Adhesive ts Bond the Tube Together |
| Vistamaxx 6102 (PP) + | Low Cost Foam − |
| 2%-10% Expandable | Good Elastic Properties |
| Microspheres (Akzo 950 MB 80) | |
| Bynel 3860 | Tie Layer to Bond TPU to the Core |
| Elastollan 1170A TPU | High Elastic Recovery + |
| | Enables RF Welding |

In one embodiment, the blown film and foam are of Lubrizol's Pellethane P86A, with expandable microspheres used as the primary foaming agent for the blown thermoplastic urethane TPU core layer(s). Blown thermoplastic urethane (TPU) foam provides cost-effective layer construction with good mechanical properties (tensile and recovery). When applied in compression braces and supports stiffer foam laminates that are perforated allow for a thinner overall laminate to provide an equal amount of compression.

In one embodiment, the core layer has a thickness of less than about 0.065 inches, less than about 0.055 inches, less than about 0.045 inches, less than about 0.035 inches, less than about 0.025 inches, less than about 0.015 inches, and less than about 0.010 inches. The thickness of the core layer is measured at its rest state and prior to any compression or displacement by welding.

In one embodiment, at least one of the main body and the strap includes additional layers to provide various functions, including, but not limited to: comfort, softness, breathability, elasticity, and stiffness. For example, the main body and strap can include a liner positioned to interface with the body part when in use to provide a softer feel to the user. Additional layers can include, but are not limited to: fabrics, non-wovens, fastening hardware like buckles and slides, hook and loop components, padding, foams, flexible stays, rigid or stiffening components, heating or cooling elements, or various sensors. In one embodiment, the core layer can be RF welded or heat sealed to the other layers. This can allow for unique patterns to not only bond the materials together but can also provide extra rigidity to areas where additional support is needed. In one embodiment, the core layer has a fabric positioned on both of its main surfaces, forming a tri-layer laminate.

The main body of the brace includes a first zone having a first specific tensile response and a second zone having a second specific tensile response. While the specification describes the brace as including two different zones of specific tensile response, the brace may include any number of zones of specific tensile response without departing from the intended scope of the present invention, as long as there are at least two zones. The varying zones provide support while also providing increased comfort and fit. The first and the second zones of specific tensile response can be produced by various methods. In a first method, various layers can be paired with the core layer. In a second method, the effective area under tension of the core layer can be modified. In a third method, the density of the weld area of the main body can be altered to create the differing zones. In the second and third methods, the first and second zones of specific tensile response are formed from the same materials and fabrics.

In the first method, the brace is formed of different fabrics having varying attributes to help provide different zones of specific tensile response that result in different levels of support, fit, and comfort. By varying the fabric layers that are laminated in each zone, differing zone properties can be created. For example, zones which combine a perforated core with elastic fabrics allow air permeability, flexibility, and stretch. A contrasting example is pairing a perforated core with an inelastic fabric resulting in zones of high tensile stiffness without substantially sacrificing air permeability.

The second method of creating different zones of specific tensile response is by modifying the effective area of the core layer that is under tension. Restriction of movement or immobilization of a body part, such as a joint, can be accomplished by wrapping the body part using inelastic materials. However, elasticity and compliance are critical to fit and comfort of the brace. When the brace uses materials having low elasticity as the core layer for support, means for compliance or stretch must be added to the core layer to accommodate movement and conformation to the body part. In the second method of creating different zones of specific tensile response, the core layer can be cut and patterned in order to provide varying areas of compliance, compression, elasticity, and support.

To accommodate fit across a large range of body part sizes, portions of the braces can have more or less elasticity. For example, the straps may be designed to have more elasticity to increase the adjustability of the brace. To help achieve this elasticity, the stiffness of the core layer can be modified by removing (i.e., perforating) or disabling (i.e., selective slitting) areas from specific areas of the core layer. The stiffness properties of the core layer are dependent on the orientation of tension and geometry of material removal. The tensile performance of different zones can thus be defined as a function of material removed/disabled within the core layer. Thus, by removing or disabling areas of the core layer, the effective area under tension can be modified.

In selective slitting, no material is removed from the core layer, rather, the areas bound by the slit lines do not contribute to tensile response. Generally, high slit length density areas have lower stiffness with high elongation and conformability and low slit length density areas have higher stiffness. In addition to reducing the effective area, the slits can also function to add permeability, marginally decrease in-line stiffness, reduce perpendicular stiffness, and reduce bunching at a flexion point, such as at a joint. In one embodiment, the slits form a pattern. The slits pattern may be a gradient of slit lengths to change specific area performance of the brace at a specific area. For example, the gradient may allow for increased conformability and softness around bony prominences, and without significantly reducing stiffness along lines of non-extension on the body. In one embodiment, the slits form an auxetic pattern or an expanding pattern. The expanding pattern provides increased breathability and potentially improved perceived quality (i.e., does not thin-down when stretched) when worn. In one embodiment, the slits can be manufactured using conventional steel rule dies in a “clicker-press”. Steel rules may be notched to the corresponding pattern.

The effective area of the core layer under tension can be modified while keeping the core layer composition, welding (tool area and pressure) and fabric pairings constant. By modifying the effective area of the core layer that is under tension, different zone performances can be created. While varying force can be applied to result in a broad range of elongations, at elongations of over 60%, the fabric tensile properties can dominate the response of the body. In one embodiment, at 50% elongation, modifying effective area can change the force response by at least about 7%, at least about 10%, at least about 15%. In one embodiment, at 25% elongation, modifying effective area can change the force response by about 12%, at least about 25%, at least about 32%.

The third method of creating different zones of specific elongation is by modifying the density of the weld area. The density of the weld area can be modified by any method known to those of skill in the art. For example, the density can be modified by changing the diameter of the pin heads used to create the voids in the mold or by changing the amount of the area separating adjacent voids. The tensile performance of different zones can be defined as a function of the weld area of the laminate. Generally, as the density of the weld area increases, the force at specified tensile also increases. In one embodiment, at 50% elongation, various weld densities required different forces. In one embodiment, at 25% elongation, lower weld area densities have minimal effects on the force.

### EXAMPLES

Objects and advantages of this disclosure are further illustrated by the following comparative and illustrative examples. Unless otherwise noted, all parts, percentages, ratios, etc. in the examples and the rest of the specification are by weight. The following abbreviations are used herein: lbf=pounds force, cm³/cm²/sec=cubic centimeters per square centimeter per second, in=inches, cm=centimeters, mm=millimeters, μm=micrometers=10′ meters, min=minutes, mils=0.001 in, rev/min=revolutions per minute, ° C.=degrees Celsius, PSI=pounds per square inch, kN=kiloNewtons, R.H=relative humidity.

### Sample Preparation

TABLE 1
Materials

| Material | Source | Function |
|---|---|---|
| ELASTOLLAN | BASF, Ludwigshafen, | Thermoplastic |
| 1170A TPU | Germany | polyether- |
| | | polyurethane |
| | | elastomer |
| | | (TPU) |
| BYNEL 3860 | Dow, Midland, MI, US | Anhydride- |
| | | modified ethylene |
| | | vinyl acetate |
| | | (EVA) adhesive |
| | | resin tie layer |
| PLEXAR | LyondellBasell, | Anhydride- |
| PX1164 | Rotterdam, Netherlands | modified ethylene |
| | | vinyl acetate |
| | | (EVA) resin |
| | | tie layer |
| VISTAMAXX | ExxonMobil, Irving, | Propylene-based |
| 6102 | TX, US | elastomeric |
| | | polymer |
| | | foam matrix |
| EXPANCEL | Nouryon, Amsterdam, | Expandable |
| 950 MB 80 | Netherlands | microspheres |
| VERSAFLEX PF | PolyOne, Avon Lake, | Extrudable |
| MD6748 | OH, US | Pressure- |
| | | Sensitive |
| | | Adhesive (PSA) |
| W1 Mesh | Cathaya Fabrics Cathaya | Mesh Fabric |
| | Health Care, Hangzhou, | |
| | Zhejiang, China | |
| WeftKnit- | Cathaya Fabrics Cathaya | Lining Fabric |
| 20190927 | Health Care, Hangzhou, | |
| | Zhejiang, China | |
| Nylon Unbroken | VELCRO NORTH | UBL Fabric |
| Loop 3368, | AMERICA Manchester, | |
| part number | NH, USA | |
| 127693 | | |
| Polyester SP- | Sportek International Inc, | Lining Fabric |
| XP18 | Commerce, CA, US | |
| Nylon-Spandex | Cathaya Fabrics Cathaya | Elastic UBL |
| UBL 0-1 | Health Care, Hangzhou, | Fabric |
| | Zhejiang, China | |
| Nylon Unbroken | Cathaya Fabrics Cathaya | UBL Fabric |
| Loop | Health Care, Hangzhou, | |
| | Zhejiang, China | |
| ESTANE MVT | Lubrizol, Wickliffe, | Thermoplastic |
| 75AT3 | OH, US | polyether- |
| | | polyurethane |
| | | elastomer (TPU) |
| DUALITE U0 | Chase Corporation, | Expandable |
| 10-185D | Westwood, MA, US | microspheres |
| ELVAX 420 | Dow, Midland, MI, US | Ethylene Vinyl |
| | | Acetate (EVA) |
| | | copolymer |
| A-C 400AC | Honeywell, Charlotte, | Ethylene Vinyl |
| | NC, US | Acetate (EVA) |
| | | copolymer |
| STRUKTOL | AB Chem Inc, Dorval, | fatty ester |
| TR044 | QC Canada | processing aid |

### Examples 1-3

Core Layer: The core layer (a blown film composite) for Examples 1-3 was prepared as follows. A seven-layer film was produced using a seven-layer annular stack die (obtained under the trade designation “COEX 7-LAYER” (Type LF-400) from Labtech Engineering, Samutprakarn, Thailand). Airflow to the die was manually controlled to achieve a blow-up ratio of about 2:1. The bubble was subsequently collapsed about ten feet above the die and rolled up. The feed materials were supplied by 7 independent 20 mm diameter extruders, each with about a 30:1 length to diameter ratio. A first extruder was used to melt and extrude the VERSAFLEX PF MD6748 PSA into an inside channel of the annular stack die. A screw speed of 30 rev/min was used. The melt temperature was maintained at 180° C. Second, third, fourth, and fifth extruders were used to feed, to the next four channels of the annular stack die, VISTAMAXX 6102 elastomer and a masterbatch pellet containing a 65% concentration of EXPANCEL 950 MB 80 expandable microspheres. The blend ratio was maintained at 94% of the propylene-based elastomer and 6% of the microsphere masterbatch. Extruder speeds were maintained at 60 rev/min. A sixth extruder was used to feed BYNEL 3860 adhesive resin. A screw speed of 30 rev/min was used. The melt temperature was maintained at 204° C. A seventh extruder was used to feed ELASTOLLAN 1170A TPU to the outside channel of the annular stack die. A melt temperature of 204° C. was maintained. The extruder speed for this resin was maintained at 80 rev/min. Because the bubble was subsequently collapsed, and the innermost layer of the film was a pressure-sensitive adhesive, the finished film, after edge trimming of the collapsed bubble, was in effect a seven layer film where the outermost (or “skin”) layers were the TPU, layers 2 and 6 were tie layers, the center layer (layer 4) was the result of the joining of two layers of the pressure sensitive adhesive, and layers 3 and 5 were each the product of the merging, while in the melt, of seven original layers of the propylene based elastomer with expandable microspheres. The blown film foam core layer was 19-24 mils (0.48-0.61 mm) thick.

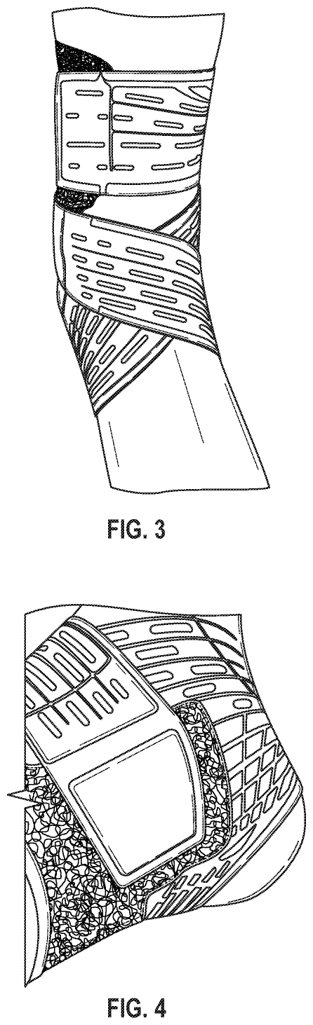

Perforation: The core layer material was laser perforated prior to fabric lamination to modify tensile behavior and increase air permeability with a VLS6.6 laser cutting system (Universal Laser Systems, Inc., Scottsdale, Ariz., US). Samples were ablated with a 10.6 μm CO₂ laser using 100% power, 48% speed, 300 points per inch, at Z-focal height of 0.025 in (0.635 mm). Material that was separated from the core during the perforation process was discarded. The illustrations in FIGS. 8(a), 8(b), and 8(c) show the laser perforation paths for Zones 1, 2, and 3, respectively. During tensile testing, test specimens were pulled along the x-axis direction as indicated by the axes shown in FIG. 8.

Lamination: The perforated core layer was placed between layers of fabric and the sandwich was bonded to create a brace having three zones with discrete tensile properties. Bonding was done using a High Frequency Generator Model No. DHL0w000 (Kabar Manufacturing Corporation, Farmingdale, N.Y., US) with power output of 10 kW at a frequency of 27.12 kHz. Other bonding parameters were adjusted as appropriate to obtain adequate bonding between layers. 1 in (2.54 cm) wide tensile test specimens were cut from each of the zones. Example 1 (Zone 1) and Example 2 (Zone 2) consisted of the perforated core layer laminated between a layer of W1 mesh fabric and a layer of WeftKnit-20190927 lining fabric. Example 3 (Zone 3) was the perforated core material laminated between VELCRO nylon 3368 UBL fabric and WeftKnit-20190927 lining fabric.

### Examples 4-7

Core Layer: The core layer for Examples 4-7 was prepared similarly to the core layer for Examples 1-3, with the following exceptions. The first extruder was used to melt and extrude ESTANE MVT 75AT3 TPU into an inside channel of the annular stack die using a screw speed of 60 rev/min. The melt temperature was maintained at 180° C. The second, third, fourth, fifth and sixth extruders were used to feed, to the next five channels of the annular stack die, a ESTANE MVT 75AT3 TPU and a masterbatch pellet of DUALITE U0 10-185D expandable microspheres dispersed in ELVAX 420 and A-C 400AC EVA copolymers and STRUKTOL TR044 processing aid at a 54% concentration of expandable microspheres using a twin screw process as commonly known in the art. The blend ratio was maintained at 90% of the TPU and 10% of the microsphere masterbatch. Extruder speeds were maintained at 45 rev/min. The seventh extruder was used to feed ESTANE MVT 75AT3 TPU to the outside channel of the annular stack die. A melt temperature of 180° C. was maintained. The extruder speed for this resin was maintained at 60 rev/min. The blown film foam core layer was 32-35 mils (0.81-0.89 mm) thick.

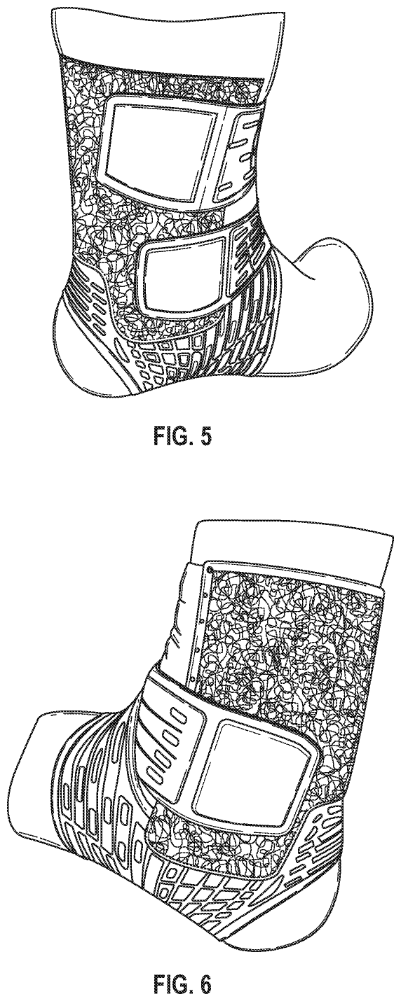

Perforation: Prior to fabric lamination, core layers were perforated using the laser perforation technique described earlier for Examples 1-3. For Examples 4-7, the core material was slit to selectively reduce the cross-sectional effective area under stress in tension, as shown in FIGS. 9(a)-(d), which depict pairs of “C” shaped slits having various height. Table 2 lists the effective area under tension for Examples 4-7 (which correspond to FIGS. 9(a)-(d), respectively.) Although the areas enclosed by the slits was not detached from the sample, those areas were not considered to contribute to the tensile strength of the material. During tensile testing, test specimens were pulled along the x-axis direction as indicated by the axes shown in FIG. 9.

TABLE 2
Effective Area for Examples 4-7

| | Example | Example | Example | Example |
|---|---|---|---|---|
| | 4 | 5 | 6 | 7 |
| Effective Area | 17% | 25% | 50% | 100% |
| Under Tension | | | | |

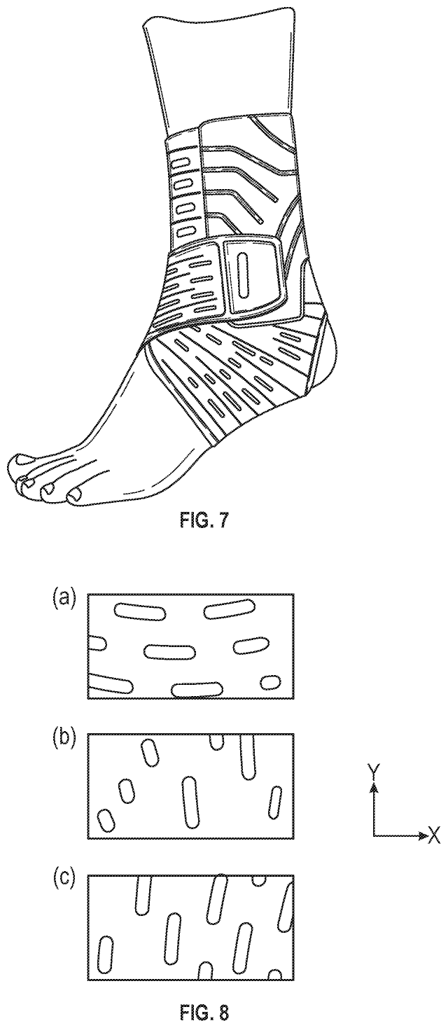

Lamination: Samples were edge-sealed manually with a PACKRITE Model RHS1-F heat sealer (Mettler Toledo Company, Worthington, Ohio, US). The core material is sandwiched between Polyester SP-XP18 lining fabric and nylon-spandex UBL 0-1 fabric, inserted into a holding fixture and heat sealed at 190° C. for 7.0 seconds under 60 psi, with a 0.020 in (0.51 mm) seal gap per side. Each test specimen was sealed on 2 sides as indicated in FIG. 10.

### Comparative Examples CE1 and CE2

CE1 was of a layer of polyester SP-XP18 lining fabric only, and CE2 was a layer of Cathaya Nylon-Spandex UBL 0-1 UBL fabric only.

### Examples 8-10

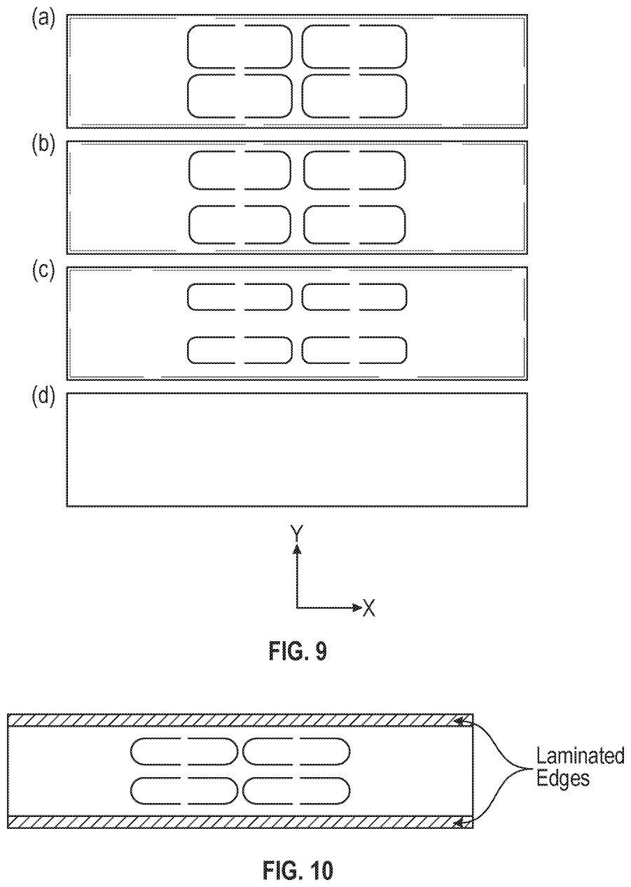

The core layer for Examples 8-10 was prepared as described above for Examples 1-3, except that the sixth extruder was used to feed PLEXAR PX1164 EVA resin as the tie layer. Examples consisted of unperforated core layer laminated between a layer of nylon 3368 UBL fabric and a layer of SP-XP18 lining fabric. Fabric samples were cut by hand into 1.5 in×8 in (3.8 cm×20.3 cm) rectangles, and the core layer was cut with a clicker press into 1 in×8 in (2.5 cm×20.3 cm) samples. Samples were layered as described above, and the sandwich was placed between a 1 in×6 in (2.5 cm×15.2 cm) solid aluminum block and a 1 in×6 in (2.5 cm×15.2 cm) aluminum block (shown in FIG. 11) having 125 equally spaced 0.125 in (0.32 cm) holes that accept a 0.205 in (0.52 cm) diameter head pin with a 0.125 in (0.32 cm) stem. Varying the amount of the head pins varied the surface weld area from 68.8% (Example 10, 100% of holes filled) and 34.4% (Example 9, 50% of holes filled). The parts were bonded using a High Frequency Generator Model No. DHL0w000 (Kabar Manufacturing Corporation, Farmingdale, N.Y., US) with power output of 10 kW at a frequency of 27.12 kHz. The power set point of the Kabar High Frequency Generator was 0.56 Amps for Example 9 and 0.65 Amps for Example 10, and the remaining welding parameters are provided in Table 3. Example 8 was not welded (i.e., had 0% weld area). For FASE testing of Example 8, the three strips of material were layered and loaded into the jaws of the Instron testing machine with the core layer sandwiched between the polyester SP-XP18 fabric and the nylon 3368 UBL fabric.

TABLE 3
Welding Parameters for Examples 8-10

| | | | |
|---|---|---|---|
| Pre-Seal Time | 1.0 sec | PC Position | 40 |
| Step Start Time | 0.3 sec | PC Slowdown | 60% |
| Seal Time | 7.0 sec | Power Set Point | see text |
| Cool Time | 1.0 sec | Max RF Power | 1.00 Amps |
| Time to Set Point | 0.1-7.0 sec | PSI | 60 PSI |

### Test Methods

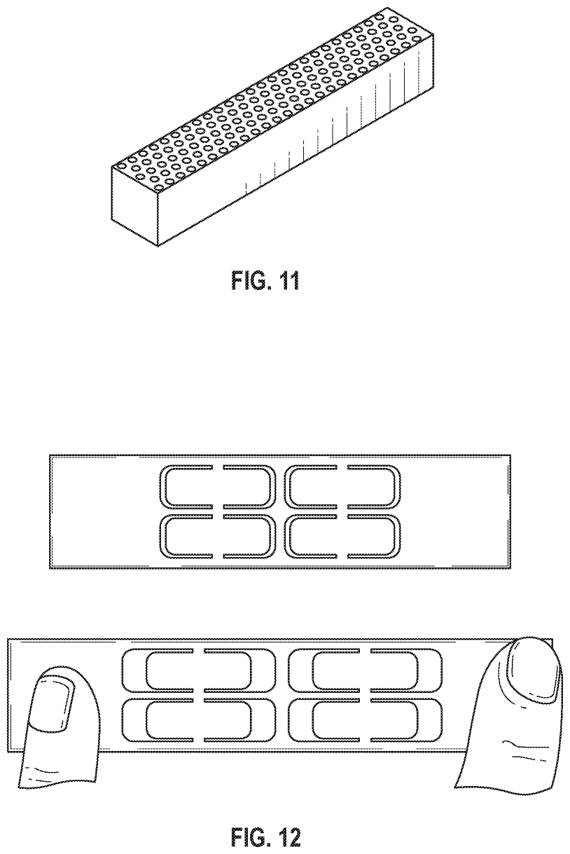

Force at Specified Elongation (FASE): Tensile force at various values of elongation were performed on using an INSTRON constant rate of extension (CRE) machine (Instron, Norwood, Mass., US). A 2 kN load cell was used with a separation speed of 5 in/min (12.7 cm/min). A rectangular strip of elastic material was placed in the tester and stretched to 50% elongation then relaxed three times. The third cycle was plotted and values of tension force at 25%, 30%, and 50% elongation were recorded. Prior to testing, samples were conditioned for 16 hours in standard environment (23+/−1° C., 65+/−2% R. H.). Test specimens were cut using hydraulic press with steel rule die to produce 1 in (2.54 cm) wide strips of various materials. Except where noted, the grip gap for test specimens was 2 in (5.08 cm). Examples 1-7 were elongated in the x-axis direction indicated by the axes in FIGS. 8 and 9. FIG. 12 (bottom) shows a specimen of Example 5 under tension to illustrate the direction of testing. FIG. 12 (top) shows a specimen of Exmaple 5 after FASE testing has been complete. Test specimens were tested in triplicate and the results averaged for sample.

Air permeability: To evaluate the breathability of the fabric or fabric laminates, air permeability was determined by testing samples on a FX3300 Air Permeability Tester III (TEXTEST Instruments, Schwerzenbach, Switzerland) according to ASTM D737-18, “Test Method for Air Permeability of Textile Fabrics.” Breathability was measured by adjusting the rate of air flow passing perpendicularly through a known area of material to obtain a prescribed air pressure differential between the two fabric surfaces.

### Results

Table 4 lists the tensile force at 50% elongation and air permeability for Examples 1-3. Examples 1 and 2, which were identical except for having different perforation patterns, exhibit approximately the same tensile load but have different values of air permeability. Example 3, which was made with relatively inelastic nylon 3368 UBL fabric, demonstrates a significantly different tensile force, without substantially sacrificing the air permeability (breathability.)

TABLE 4
Air Permeability and Tensile Force at 50% Elongation

| | Example 1 | Example 2 | Example 3 |
|---|---|---|---|
| Tensile force at | 3.741 | 3.830 | 35.955 |
| 50% elongation, | | | |
| lbf | | | |
| Air Permeability, | 5.430 | 14.175 | 9.059 |
| cm³/cm²/sec | | | |

Table 5 summarizes the tensile force at 25% and 50% elongation for a series of examples having the same core material composition and welding structure and demonstrates the effect of effective area of the core layer that is under tension. Individual fabric responses are included to show that fabrics contribute increasing amounts of force to the trilaminate performance at higher elongations. At 50% elongation, modifying the effective area changed the force response by approximately 7%. At 25% elongation, modifying the effective area changed the force response by approximately 12%.

TABLE 5
Effect of Effective Area on Tensile Force

| | Tensile force | Tensile force |
|---|---|---|
| | at 25% | at 50% |
| | elongation, lbf | elongation, lbf |
| Comparative Example CE1 | 0.148 | 0.328 |
| Comparative Example CE2 | 0.159 | 1.221 |
| Example 4 | 0.901 | 2.897 |
| Example 5 | 1.019 | 3.130 |
| Example 6 | 1.841 | 5.068 |
| Example 7 | 4.10 | 9.89 |

Table 6 summarizes tensile performance as a function of the weld area of laminate Examples 8-10. As the density of the weld area increased, the force at specified tensile elongation also increased. Table 6 shows that Example 9 and Example 8 required a similar tensile force to achieve 25% elongation, demonstrating that a weld area of 34.4% had minimal effect on the tensile force. A larger difference in the tensile force at 50% elongation was observed among Examples 8, 9, and 10 was observed.

TABLE 6
Effect of Weld Area on Tensile Force

| | Tensile force at | Tensile force at |
|---|---|---|
| | 25% elongation, lbf | 50% elongation, lbf |
| Example 8 | 5.2 | 10.1 |
| Example 9 | 4.7 | 12.1 |
| Example 10 | 6.3 | 22.2 |

Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows. All references cited in this disclosure are herein incorporated by reference in their entirety.

What is claimed is:

1. A brace comprising:
a main body; and
a strap attached to the main body for adjusting the main body around a body part,
wherein the main body and the strap are a unitary body,
wherein the brace includes a first zone having a first specific tensile response and a second zone having a second specific tensile response.

2. The brace of claim 1, wherein the brace is formed of a polymeric material.

3. The brace of claim 1, wherein the brace has a thickness of no greater than about 0.065 inches.

4. The brace of claim 1, wherein when the brace is subjected to 50% elongation, a difference between a force response of the first specific elongation and a force response of the second specific elongation is at least about 7%.

5. The brace of claim 1, wherein the first zone and the second zone are formed from different materials.

6. The brace of claim 1, wherein the first zone and the second zone are formed from the same materials.

7. The brace of claim 1, wherein the first zone and the second zone have different permeabilities.

8. The brace of claim 7, wherein the different permeabilities of the first zone and the second zone are due to one of removing portions of the brace or slitting the brace.

9. A brace comprising:
a body having a core layer; and
a strap attached to the body,
wherein the body includes a first zone having a first specific elongation and a second zone having a second specific elongation, and
wherein the core layer has a thickness of no greater than about 0.065 inches.

10. The brace of claim 9, wherein when the brace is subjected to 50% elongation, a difference between a force response of the first specific elongation and a force response of the second specific elongation is at least about 7%.

11. The brace of claim 9, wherein the first zone and the second zone are formed from different materials.

12. The brace of claim 9, wherein the first zone and the second zone are formed from the same materials.

13. The brace of claim 9, wherein the first zone and the second zone have different permeabilities.

14. The brace of claim 13, wherein the different permeabilities of the first zone and the second zone are due to one of removing portions of the brace or slitting the brace.

15. The brace of claim 14, wherein slitting the brace comprises slitting the brace to form a pattern.

16. The brace of claim 15, wherein the pattern is an expanding pattern.

17. The brace of claim 9, wherein the body and the strap are a unitary piece.

18. The brace of claim 9, wherein the core layer has a thickness of no greater than about 0.035.

19. The brace of claim 9, wherein the core layer is formed of a polymeric material.
